(19) United States
(12) Patent Application Publication
(10) Pub. No.: US 2025/0352741 A1
(43) Pub. Date: Nov. 20, 2025

(54) Smart Inhaler

(71) Applicants: Pavan MUTTIL, Albuquerque, NM (US); Christian SLOUGH, Albuquerque, NM (US); Amelia BIERLE, Albuquerque, NM (US); Dominic THOMPSON, Albuquerque, NM (US); Nathan JACKSON, Albuquerque, NM (US); UNM Rainforest Innovations, Albuquerque, NM (US)

(72) Inventors: Pavan Muttil, Albuquerque, NM (US); Christian Slough, Albuquerque, NM (US); Amelia Bierle, Albuquerque, NM (US); Dominic Thompson, Albuquerque, NM (US); Nathan Jackson, Albuquerque, NM (US)

(73) Assignee: UNM Rainforest Innovations, Albuquerque, NM (US)

(21) Appl. No.: 18/867,724

(22) PCT Filed: May 22, 2023
(86) PCT No.: PCT/US2023/023156

§ 371 Date: Nov. 20, 2024

Related U.S. Application Data
(60) Provisional application No. 63/344,294, filed on May 20, 2022.
(60) Provisional application No. 63/417,902, filed on Oct. 20, 2022.
(60) Provisional application No. 63/432,938, filed on Dec. 15, 2022.

(51) Int. Cl. A61M 15/00

(52) U.S. Cl. CPC A61M 15/0001; A61M 15/0028; A61M 15/0091; A61M 2205/3303; A61M 2205/3306; A61M 2205/3327; A61M 2205/3334; A61M 2205/3365; A61M 2205/50; A61M 2230/43

(57) ABSTRACT

A real-time monitoring, fully integrated (no add-on device required), smart inhaler (SI) that will allow objective measurements, such as spirometry, peak inspiratory flow rate, and breath NOx (exhaled nitrogen oxide) levels.

### RELATED APPLICATIONS

This application is a US 371 National Phase of PCT/US2023/023156 filed on 22 May 2023, which claims priority to U.S. Provisional Application Nos. 63/432,938 filed 15 Dec. 2022; 63/417,902 filed on 20 Oct. 2022 and 63/344,294 filed 20 May 2022, all of which are incorporated herein in their entirety

### BACKGROUND OF THE INVENTION

Asthma remains a serious chronic inflammatory disorder of the airways for two primary reasons: poor treatment adherence and sub-optimal technique of using inhalers. In the US, only between 30 and 70% of asthma patients adhere to their medications; importantly, poor treatment adherence is responsible for up to 66% of hospitalizations. Additionally, asthma patients also have poor inhaler-use techniques that can be attributed to reasons such as cognitive decline and forgetting their inhaler-use training during long-term use. The combination of irregular (poor adherence) and ineffective (poor technique) inhaler use are prevalent in 80% of asthmatic patients leading to high treatment costs and suboptimal health outcomes.

Nonadherence to medication and poor inhaler technique drive suboptimal clinical outcomes: Adherence is defined as the extent to which patients take medications as prescribed by their health care providers. Nonadherence to medication is common in diseases that require life-long treatment. In the US, adherence to asthma medication is between 30 and 70 percent and up to 66 percent of medication-related hospitalizations are due to poor treatment adherence. Nonadherence to asthma medication is a leading contributor to high treatment costs and suboptimal health outcomes. A critical component of adherence is poor inhaler technique. Improper inhaler technique leads to a discrepancy between attempted adherence and actual adherence. Irregular (poor adherence) or ineffective (poor technique) inhaler use occur in 80 percent of asthmatic patients.

Smart Technology to improve asthma adherence: Smart technology has revolutionized how chronic diseases are treated. The introduction of continuous glucose monitors (CGMs) has significantly improved blood glucose values for diabetic patients. Smart inhalers (SI) were introduced to improve both inhaler technique and treatment adherence. Current SI achieve adherence by reminding patients to take their medication on time or by having dose counters. SI have also been combined with external devices that assess inhaler technique using audio prompts based on the patient's flow pattern. However, existing SI have only moderately improved treatment adherence. They generate data that overburden health care providers and does not address interoperability concerns. An interoperability platform will allow patients to easily access their healthcare information and also empower them on how to use it correctly. Current SI designs do not attempt to improve treatment adherence using gamified incentives. SI need to include tools that are patient-centered and go beyond data collection, generating real-time feedback for patients using improved technology.

Measuring biomarkers in exhaled breath condensate: Inflammatory and oxidative changes in the lung are indicative of worsening pathophysiology and disease severity in asthma patients. Compounds such as nitric oxide (FeNO; volatile) and nitrogen oxides (NOx; non-volatile) have been used as markers in a patient's exhaled breath condensate (EBC). FeNO was the first useful non-invasive marker of airway inflammation in asthma and still is the most widely used. Further, FeNO monitoring in patients with a history of exacerbations is associated with a substantial reduction in asthma-related inpatient hospitalization or emergency department claims and charges. These findings demonstrate that FeNO monitoring in asthma management is associated with significant cost savings. FeNO alone is not sufficient but coupling FeNO monitoring with standard clinical tools (history, physical examination, and lung function tests) offers an improvement to disease control.

Interventions to improve adherence in asthma: There is insufficient research into intervention to improve adherence to asthma treatment regimens. Existing intervention studies have methodological limitations and biases that prevent definitive conclusions about the effect these interventions could have on asthma control. There is a need to develop better intervention programs to improve medication adherence for chronic diseases. Such programs will have a larger effect on the health outcomes of patient populations than any new or improved treatment. Generally, intervention strategies to improve medication adherence fall into four major categories: patient education, improved dosing schedules, increased clinic hours to reduce wait times, and improved communication between physicians and patients. To fully embrace a patient-centered approach, all factors relating to adherence must be integrated into any intervention plan. In treating chronic diseases, the health care system cannot succeed by focusing on specific components, but rather must recognize the interconnectedness of cognitive factors, interpersonal factors, patient satisfaction, patient attitudes, and cultural variations.

Role of gamified incentives and behavioral economics to improve adherence: Recent developments in incentive strategies and gamification to improve medication adherence have yielded positive results. Gamified incentives take advantage of the human tendency to be present-biased, where individuals settle for a smaller reward in the present rather than waiting for a larger reward in the future. Present-bias is also observed in medication adherence because it can take up to six weeks for asthma patients to feel the benefit of their medications. These patients incur adherence costs in the present but do not reap the benefits of adherence until far in the future. Numerous laboratory and field studies have shown that individuals have difficulty making wise choices regarding their treatment options. Incentives can be used to motivate individuals to perform specific tasks in exchange for a reward. Gamification works as one of the tools for motivation, giving patients the freedom to create their adherence pathways by providing patient autonomy through the use of goals, challenges, rules, interactions, and rewards. The gamification of patient-centered incentive designs can offset the physical and mental costs that individuals undergo when attempting to change their health behaviors.

Role of choice architecture for educating patient behavior: The growing field of choice architecture research investigates how an individual's decision-making capability can influence their behavioral choices over other alternatives. Choice architecture research has gained recent attention for its potential to apply insights from behavioral research to areas in marketing, policy-making, development aid, and healthcare. This field was triggered by Thaler and Sunstein's publication Nudge, in which they suggested that predictable deviations from rational behavior can be used to “nudge” people into socially desirable directions including health. Choice architecture refers to the concept that changes made in the environment of an individual can affect their decision-making ability and behavior while also empowering them by preserving freedom of choice. Such an approach alters people's behavior predictably without threatening their choices or changing their economic incentives. Insights from choice architecture can optimize intervention programs to improve medication adherence.

### BRIEF SUMMARY OF THE INVENTION

In one embodiment, the present invention concerns a real-time monitoring, fully integrated (no add-on device required), smart inhaler (SI) that will allow objective measurements, such as spirometry, peak inspiratory flow rate, and breath NOx (exhaled nitrogen oxide) levels.

In other embodiments, the present invention uses to provide an interface between the SI and a mobile device.

In other embodiments, the present invention incorporates a behavior change frameworks utilizing patient-centered gamification, behavioral economics and patient incentives to improve medication adherence.

In other embodiments, the present invention, employs patient-centered approaches such as gamification (to overcome present-bias) and choice architecture (empowering patients by making changes to their environment) to improve treatment adherence.

In other embodiments, the present invention uses a SI in combination with behavioral economics-incentive design and gamification to improve the treatment adherence of asthmatic patients.

In other embodiments, the present invention concerns an inhaler designed to measure inhaler usage, peak inspiratory flow rate, and patient (breath) NOx levels.

In other embodiments, the present invention concerns an inhaler designed to achieve optimal weight, power usage, and ease of manufacture at a limited scale.

In other embodiments, the present invention concerns an inhaler using a wireless interface between the SI and external devices such as cell phones and external databases.

In other embodiments, the present invention concerns an app that will allow the transmission of stored data in a SI device via Bluetooth.

In other embodiments, the present invention concerns an inhaler that will allow data integration with a smartphone app and provide relevant information to patients, physicians, and researchers.

In other embodiments, the present invention concerns gamification methods that empower patients to create adherence avenues by providing patient autonomy through the use of goals, challenges, rules, interactions, and rewards. This allows asthma patients to make better decisions regarding their treatment adherence.

In other embodiments, the present invention concerns a digital intervention that reinforces medication adherence habits that will be implemented into an app.

In other embodiments, the present invention improves inhaler-use techniques from patients through feedback mechanisms and improving the tools aimed to objectively track disease exacerbation and patient compliance to medication.

In other embodiments, the present invention concerns a SI system adapted to collect data for physicians and patients, allowing them to understand each patient's unique asthma profile. Since multiple players contribute to the successful healthcare outcome for patients, interoperability between these players becomes critical. Embodiments of the present may include interoperability in the SI system that will not only empower patients in this digital age, but also meet their evolving expectations regarding their healthcare information.

In other embodiments, the present invention overcomes difficulty in obtaining accurate and specific measures of adherence.

In other embodiments, the present invention concerns a SI that implements a multi-measure approach in which adherence is assessed through objective measures such as PIFR and NOx sensors, and subjective measures such as self-report assessments transmitted to an app.

In other embodiments, the present invention concerns digital intervention programs that improve medication adherence.

In other embodiments, the present invention concerns a SI that is capable of measuring asthma medication adherence and a digital intervention program that nudges and rewards patients for medication adherence.

In other embodiments, the present invention concerns a SI capable of accurately measuring peak inspiratory flow rate (PIFR) will provide feedback to the patient about their lung health and overall disease progression.

In other embodiments, the present invention concerns a SI having a customizable/personalized reward system has the potential to explore the mechanisms that lead to medication adherence.

In other embodiments, the present invention concerns an inhaler having an anemometer including an IR sensor that measures the rotational rotate of a fan.

In other embodiments, the present invention concerns an inhaler wherein the amount of medication being delivered may be measured by the flow rate of air through the inhaler.

In another embodiment, the present invention concerns an inhaler having a vibration motor that provides feedback to a user.

In another embodiment, the present invention concerns an inhaler comprised of an IR sensor, a fan holder unit, a fan, a mouthpiece, a ball bearing, and a body to secure the inhaler to the mouthpiece.

In another embodiment, the present invention concerns an inhaler wherein the amount of medication being delivered may be measured by the flow rate of air through a separate chamber measured by finding a fan's rate of revolution with an infrared transponder and receiver.

In another embodiment, the present invention concerns an inhaler comprised of RGB LED light and vibration motor, a centralized main body detachable from the mouthpiece, and a complete built-in assembly including the power source and all necessary electronic components.

In another embodiment, the present invention concerns an inhaler having an IR signal sender and transponder module which measures the fan's rate of revolution that correlates to the patient's ability to draw air into the lungs while using the inhaler and measures the amount of medication being delivered to the lung. In another embodiment, the present invention concerns an inhaler comprised of an IR Sensor and RGB LED assembly, vibration motor assembly, battery and switch assembly, battery door assembly, motherboard assembly, medicine holder and motherboard retention assembly, fan assembly, and mouthpiece assembly.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 is an isometric view of an embodiment of the present invention.

FIG. 2 is a side view of an embodiment of the present invention.

FIG. 3 is another isometric view of an embodiment of the present invention with portions removed.

FIG. 4 is a partial front view of an embodiment of the present invention.

FIG. 5 is an exploded view with portions removed of an embodiment of the present invention

FIG. 6 is a cross-sectional view of an embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention.

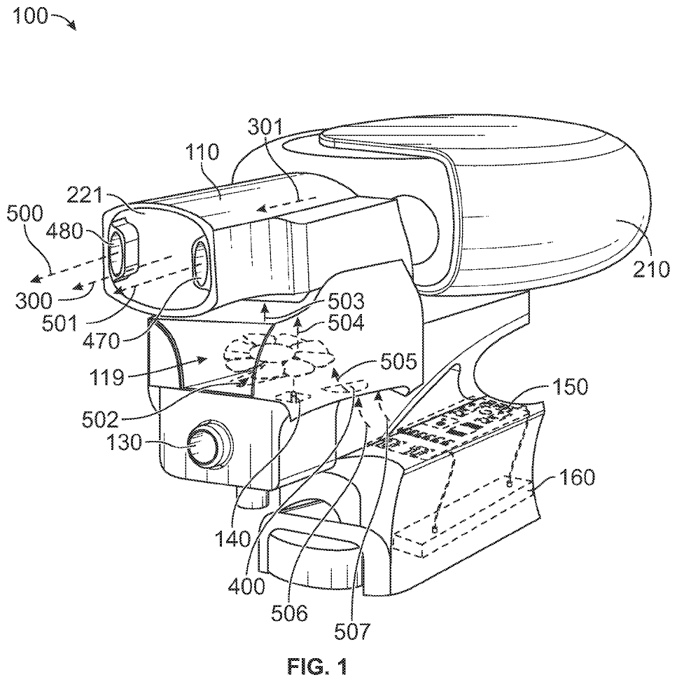

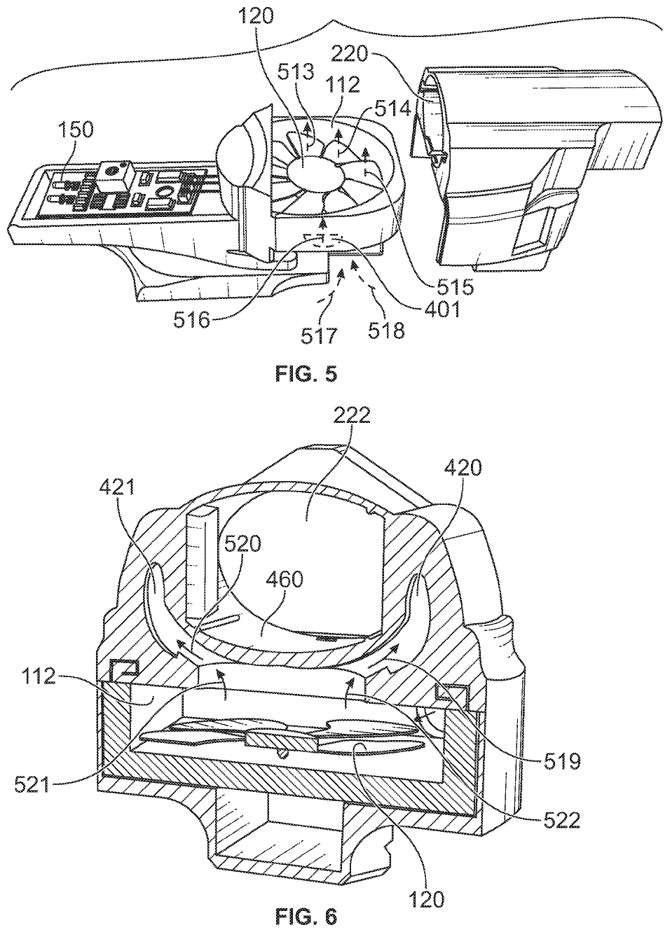

A preferred embodiment of the present is shown in FIGS. 1-6. This embodiment provides a smart inhaler system 100 that includes housing 110 having a chamber 112 in which an anemometer 119 is located. Anemometer 119 may be comprised of a fan 120, sensor 140, and circuit board 150 having a processer which together measure the rotational speed of a fan 120 along with power source 160. Clips 200 and 202 are used to releasably secure medicament container 210.

This embodiment of the present invention uses an external air-intake arrangement which provides a bifurcated air pathway system wherein a first airstream draws medication from container 210, which may contain a dry powder, for direct delivery and respiration by a patient. A second airstream is used to spin fan 120.

In a preferred embodiment the first and second airstreams are isolated from one another to avoid medicament from coating fan 120 and interfering the operation of fan 120. This is important since a laser or IR sensor 140 is used to measure the rate of revolution of fan 120 by providing feedback to circuit board 150. Fan rotation is used to calculate the strength and volume of a user's inhalation. Coating a fan with medicine will change the rate at which it spins, which in turn, changes the ability accurately calculate an inhalation and the amount of medicine delivered to a patient.

When the feedback indicates an acceptable inhalation and drug delivery, the device may be programed to active light 130. This provides a visual indicator to a user to indicate when the device is being used properly. Another means to provide feedback to a user is to use a have the device vibrate such as by the use of haptics. An audio indication may be used as well.

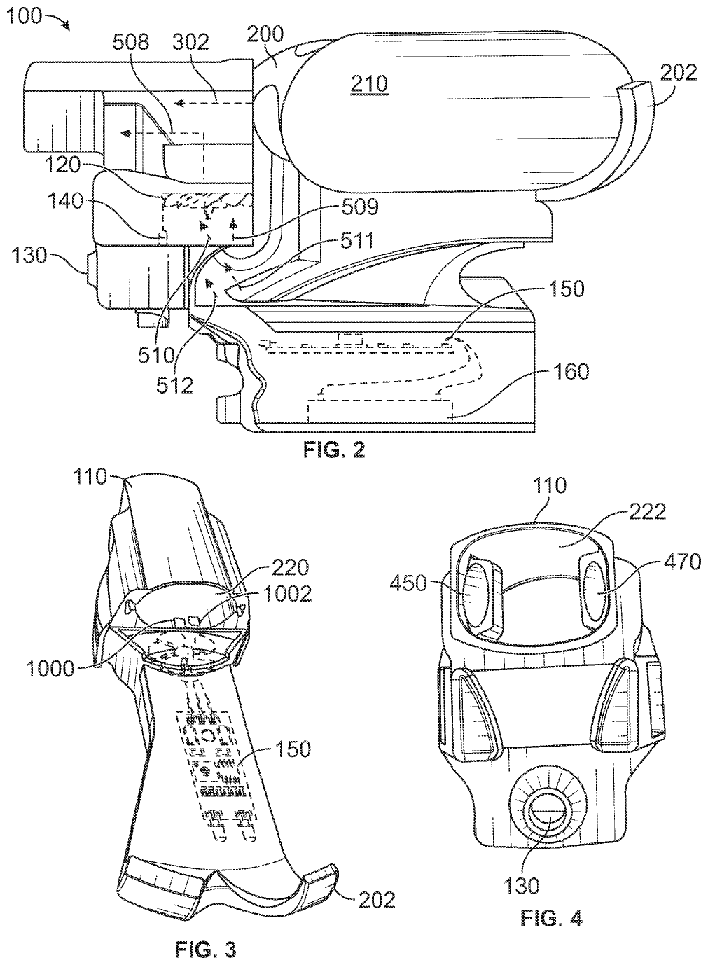

The first airstream, as shown by arrows 300-302 in FIGS. 1 and 2, is created by a user′ inhalation. When inhalation occurs, a vacuum is created which draws medicine out of container 210 into opening 220. The medicine then travels through passageway 222, as shown in FIG. 4, in housing 110 and exits opening 221 to deliver medicine to a user, as shown in FIG. 1.

Second airstream is also created by the same inhalation used to create the first airstream. When inhalation occurs, the vacuum created draws air into opening or vent 400, as shown in FIG. 1, and opening or vent 401 as shown in FIG. 6. Air is then drawn into chamber 112 and past fan 120 causing it to spin. Once past fan 120, air is directed into opposing passageways 420 and 421 which are isolated from passageway 222 by internal wall 460. Passageways 420 and 421 terminate at openings 470 and 480. Arrows 500-522 show how the second airstream travels through inhaler 100.

Because each passageway defining and airstream is a separate, closed system, taking air from the mouthpiece, where openings 221, 470 and 480 are located, and partitioning it to flow past fan 120, which is part of the inhaler's anemometer results in no pressure loss from the inhaler device. Nor is there the possibility of medication dilution.

In another embodiment, the present invention concerns a smart inhaler (SI) that improves treatment adherence and inhaler techniques. In a preferred embodiment, the SI may include: 1) incorporation of sensors to objectively measure patient parameters such as peak inspiratory flow rate (PIFR) and NOx that measure their asthma state, 2) integration with a Bluetooth-enabled connection to external devices that can be accessed by patients, healthcare providers, and policymakers, and 3) behavioral intervention strategies to improve medication adherence. In other aspects, the SI platform may utilize a patient reward system that personalizes treatment plans by integrating data generated by the user interface and patient questionnaires.

In other embodiments, the present invention incorporates a NOx sensor 1000 as shown in FIG. 3, that includes a flow-rate sensor. The NOx sensor can predict asthma exacerbation by measuring the patient's exhaled breath condensate (EBC). Inflammatory and oxidative responses in the lung are indicative of worsening pathophysiology and disease severity in asthma patients. The NOx sensor will allow for the measurement of this pulmonary biomarker.

In other aspects, a self-contained SI is provided and includes a pressure sensor 1002 that measures the patients' peak inspiratory flow rate (PIFR). The SI transmits data to a smart device. Since the airflow in the lung is restricted during an asthmatic attack, the PIFR is a valuable parameter that needs to be captured each time a patient uses their inhaler.

In other embodiments, circuit board 150 includes an ESP32 processor (NodeMCU developer board), an analog pressure sensor (+/−2000 Pa, five VDC; volts of direct current), and a battery power supply (3.3 & 5V taps). The device includes a Venturi flow measurement tap, inhaler port, spacer attachment, and mount points for electronics and sensors.

The casing for the sensors, spacer, and inhaler may be fabricated using a 3D printer (Original Prusa i3 MK3S+kit). To test the flowrate measurement of the SI, a Next-Generation impactor (NGI, Model 170 NGI, MSP Corporation, Shoreview, Minnesota) was used using clinically relevant airflow rates (30, 45, and 60 L/min).

The SI may have a precision of <5 parts per billion (ppb) or 10% of concentration; accuracy of +5 ppb or +10% of concentration, limit of detection 10 ppb, and measurement range of 10 ppb to 200 ppb.

In other embodiments, the present invention includes approaches that move beyond current health care practices, such as including data captured by mobile technology like Smartphone Apps and wearable biosensor devices. Stored data in the SI will be transmitted via Bluetooth to mobile devices, then uploaded to the cloud and compiled for analysis.

Software used with the embodiments of the invention is used to evaluate flow rate. The software takes measurements using a computer to perform a user-specified number of inhalation measurements. The software continuously monitors the sensors described above and automatically detects each inhalation as a change in the pressure difference.

Once it detects the inhalation, the present invention records and saves the pressure data. The software is optimized to measure the flow starting one second before the event and up to 10 seconds after the event. These data are saved to a file on the SI app, which then resets itself to monitor the next event. This process continues until the SI has reached the maximum number of tests.

In other embodiments, the SI of the present invention uses a low-power, BLE (Bluetooth low energy) protocol to transmit collected data to a mobile device which automatically downloads new events from the sensor whenever it is within range (˜20m). This setup will eliminate the need for internet access. If a transmitting device is unavailable when an event occurs, the sensor will store the details in its local memory and transmit the information later. The smartphone app will be capable of presenting visualized data.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. An inhaler comprising: an anemometer located in a housing; said housing having a first passageway therein and a second passageway therein; said first passageway in communication with a medicine container and configured to deliver medicine to a user during an inhalation; and said second passageway in communication with said anemometer and configured to permit air to flow pass said anemometer during an inhalation.

2. The inhaler of claim 1 wherein said first and second passageways are isolated from one another.

3. The inhaler of claim 2 wherein said anemometer includes a fan blade.

4. The inhaler of claim 3 wherein said anemometer includes a fan blade wherein a sensor and processor are used to measure the rotational speed of said fan blade.

5. The inhaler of claim 4 wherein said sensor is an infrared transponder and receiver.

6. The inhaler of claim 5 wherein said anemometer is used to measure the amount of medication being delivered.

7. The inhaler of claim 6 wherein said inhaler is configured to measure spirometry, peak inspiratory flow rate, and breath NOx (exhaled nitrogen oxide) levels.

8. The inhaler of claim 6 wherein said inhaler is configured to measure the air flow starting one second before the event and up to 10 seconds after the event.

9. The inhaler of claim 2 wherein said anemometer includes a fan blade, said fan blade located in a chamber in said housing.

10. The inhaler of claim 5 wherein said anemometer is used to measure the inspirator of a user.

11. The inhaler of claim 5 wherein said anemometer is used to measure the breathing capacity of a user.
